(19) United States
(12) Patent Application Publication
(10) Pub. No.: US 2025/0064708 A1
(43) Pub. Date: Feb. 27, 2025

(54) USE OF BOTULINUM TOXIN FOR REDUCTION OF SKIN PORE SIZE AND SEBUM PRODUCTION

(71) Applicant: MERZ PHARMA GMBH & CO. KGAA, Frankfurt am Main (DE)

(72) Inventors: Vladimir SUDIMAC, Frankfurt am Main (DE); Petra WEISSENBERGER, Frankfurt am Main (DE); Kay MARQUARDT, Frankfurt am Main (DE)

(21) Appl. No.: 18/841,985

(22) PCT Filed: Feb. 28, 2023
(86) PCT No.: PCT/EP2023/054925

§ 371 Date: Aug. 27, 2024

(30) Foreign Application Priority Data
Feb. 28, 2022 (EP) 22159241.3

(51) Int. Cl. A61K 8/66; A61Q 19/00

(52) U.S. Cl. CPC A61K 8/66; A61Q 19/00; A61Q 19/008; A61K 2800/91

(57) ABSTRACT

The present invention generally relates to the use of botulinum toxin for the reduction of skin pore size and/or sebum production using specific dosing regimens and injection schemes. Specifically, the botulinum toxin is administered by intradermal injection into the skin of a subject at a concentration of 11 U/ml to 19 U/ml and at a volume of 0.07 ml to 0.12 ml per injection point. Furthermore, the present invention relates to a method for the reduction of skin pore size and/or sebum production using said specific dosing regimens and injection schemes.

### FIELD OF THE INVENTION

The present invention generally relates to the use of botulinum toxin for the reduction of skin pore size and/or sebum production using specific dosing regimens and injection schemes. Specifically, the botulinum toxin is administered by intradermal injection into the skin of a subject at a concentration of 11 U/ml to 19 U/ml and at a volume of 0.07 ml to 0.12 ml per injection point. Furthermore, the present invention relates to a method for the reduction of skin pore size and/or sebum production using said specific dosing regimens and injection schemes.

### BACKGROUND OF THE INVENTION

In recent years, a holistic approach to use botulinum toxin not only to reduce wrinkles but also to improve skin quality has become increasingly popular. In addition, the “selfie culture” leads to more, especially younger people, with the aspiration to achieve healthy looking skin. Good skin quality is perceived as healthy and undamaged skin, which is associated with a youthful and attractive appearance.

The primary mechanism of botulinum toxin is muscle paralysis via inhibiting the exocytosis of presynaptic acetylcholine, and it is in wide-scale use for multiple cosmetic and therapeutic applications. More recently, it was found that intradermal injection of botulinum toxin is effective in skin quality improvement by decreasing facial laxity, shorten mandibular length (resulting in face lifting), reducing sebum secretion and decreasing the number and density of facial pores. These effects could be attributed to the paralysis of arrector pili muscles (pore size reduction), inhibition of acetylcholine release (decreased sebum production), and affecting the superficial layer of facial muscles (see, e.g., Park et al., Drugs Dermatol. 2021; 20(1):49-54; Li et al., J. Dermatol. Sci. 2013; 72:116-22).

Different approaches for injecting botulinum toxin intradermally are known in the art. One of them is the “mesobotox” or “microbotox” approach in which the injections are administered using hyperdiluted botulinum toxin (concentrations of less than 10 U/ml). Small volumes (20 UL and less) are administered intradermally in such manner that there are at least 15 injections points per cheek, at least 20 injection points per forehead and at least 20 to 30 injections along the jawline/in the lower portion of the face (Wu W T L. Facial rejuvenation without facelifts—personal strategies. Paper presented at: Regional Conference, in Dermatol. Laser and Facial Cosm. Surg. 2002; Sep. 13-15, 2002; Hong Kong). Often, this results in more than 100 injection points which leads to unnecessary pain for the patients, as well as discomfort and time-wasting for the injectors.

Another technique described in the literature is the “SINB” technique (Superficial Injection Needling Botulinum) reported by Calvani et al. (Plast. Surg., 2019; 27 (2):156-161). It comprises dermal injections of microdoses of botulinum toxin, not by traditional syringe but with needling technique that consists of multiple microdroplets by electrical device. The intention is to decrease sweat and sebaceous gland activity to improve skin texture. The reconstitution volume is different compared to the previously described “mesobotox” or “microbotox” approach as the resulting concentration is not hyperdiluted but concentrated (>60 U/ml).

As regards intradermal treatments aimed exclusively at improving enlarged pores and excessive sebum production, there are numerous publications with different, sometimes even contradictory data on reconstitution volume and dosage. The recommended reconstitution volumes range from 5 ml of saline solution per 100 U vial of botulinum toxin type A (BoNT/A), resulting in a concentration of 20 U/ml (see, e.g., Park et al., supra, and Shah, A. R., J. Drugs Dermatol. 2008; 7 (9):847-850) up to 3 ml of 300 U vial of BoNT/A, resulting in a concentration of 100 U/ml (see Rose and Goldberg (2012), Dermatol. Surg, 2012; 39:443-448). Specifically, Park et al. (supra) diluted 100 U of BoNT/A in 5 mL of saline solution and injected 74 points intradermally in the face (lateral face, anterior medial cheek, mandibular line, and depressor anguli oris (DAO)) for a total dose of 92 U per treatment using a 31 G needle.

Min et al. (Aesthet. Surg. J., 2015; 35 (5):600-610) propose using lower reconstitution volumes of 2.5 ml per 100 U vial, resulting in a more concentrated solution that contains 40 U/ml. Min et al. further suggest using larger injection volumes (250 UL or even 500 μL per injection point) for injection points distributed across five standard sites in the middle of the forehead. Other authors propose injecting 100 μL of saline per injection point that contains 2 U of BoNT/A and recommend that the injections are spaced out 1 cm apart from each other (Shah, supra; Li et al., supra; Sayed et al., J. Dermatol. Treat. 2021, 32 (7):771-777).

Furthermore, WO 2004/016763 A2 concerns the treatment of holocrine gland dysfunction with botulinum toxin including the reduction of excessive sebum production and pore size by intradermal injection of botulinum toxin. It discloses the treatment of excessive sebum production by injections of BoNT/A and BoNT/B into the forehead and of BoNT/B into the skin of the eyebrows, forehead, and nose and nasolabial folds. However, no clear instructions are given regarding the reconstitution volume, injection dose/volume and injection scheme for the treatment of enlarged pores and excessive sebum production.

There are several drawbacks related to the treatment of enlarged skin pore size and excessive sebum production in the prior art. The hyperdiluted approach (use of higher reconstitution ratios resulting in less concentrated solutions) results in a less pronounced effect that does not last as long, thereby leading to more frequent visits to the healthcare personnel's practices and to lower patient satisfaction. In contrast, doses that are more concentrated do not seem to provide any additional potency (Shah, supra) but may require an increase in the number of injection points, potentially leading to an increase in adverse events.

In addition, as described in Sapra et al. (J. Clin. Aesthet. Dermatol. 2017; 10 (2):34-44), the absence of standardization of the injection regimen (i.e., treating patients based on their individual needs, using treatment volumes that are neither controlled nor standardized etc.), may result in unfavorable effects. Hence, a clear guidance regarding the reconstitution volume, the injection dose and volume, and the injection scheme for the treatment of enlarged skin pores and excessive sebum production is needed.

Moreover, apart from the lack of effect which is always unpleasant for both the injector and the patient, the main concern with the use of botulinum toxin A in aesthetic indications lies in the risk of migration/spread to unintended muscles. Therefore, it is generally desired that the dosing regimen and injection scheme does not result in an unwanted toxin spread in the muscle layer located underneath the treated dermis.

Therefore, there is an ongoing need in the art for a treatment of reduction of skin pore size and sebum production which overcomes the drawbacks of the prior art, is safe and provides highly satisfying cosmetic results.

### OBJECT OF THE INVENTION

It is the object of the present invention to provide an improved botulinum toxin treatment for the reduction of skin pore size and/or sebum production that is both effective and safe.

### SUMMARY OF THE INVENTION

The present invention provides specific dosing regimens and injection schemes for the administration of botulinum toxin for the reduction of skin pore size and/or sebum production. The proposed dosing regimen, optionally in combination with the proposed injection schemes, results in a balanced and harmonious appearance of the skin on the upper ⅔ of the face, enhancing patient satisfaction without increasing side effects. More specifically, the relatively high doses will prolong the duration of effect, thereby adding to patient convenience and satisfaction, and the relatively high volume results in a certain spread necessary for achieving the desired treatment result.

In a first aspect, the present invention relates to the use of botulinum toxin for the reduction of skin pore size and/or sebum production, wherein the botulinum toxin is administered by intradermal injection into the skin of a subject at a concentration of 11 U/ml to 19 U/ml and at a volume of 0.07 ml to 0.12 ml per injection point.

Preferably, the botulinum toxin is administered at a concentration of 16 U/ml to 18 U/ml and at a volume of 0.08 ml to 0.115 ml per injection point. The injection scheme preferably comprises intradermal injection into 7 to 15 injection points, preferably 11 to 13 injection points, across the forehead, the forehead comprising the glabellar area, and into 6 to 12 injection points, preferably 9 injection points, per cheek, wherein the distance between the injection points is 0.9 cm to 1.5 cm in the cheek and 1.7 cm to 2.3 cm in the forehead, the forehead comprising the glabellar area.

In a second aspect, the present invention relates to a method for the reduction of skin pores and/or sebum production, the method comprising administering botulinum toxin by intradermal injection into the skin of a subject at a concentration of 11 U/ml to 19 U/ml and at a volume of 0.07 ml to 0.12 ml per injection point.

Preferred embodiments are set forth in the appended dependent claims and in the following detailed description taken in connection with the examples provided therein and the accompanying figures.

### BRIEF DESCRIPTION OF THE FIGURE

FIG. 1 shows an exemplary injection pattern according to the present invention for the reduction of skin pore size and/or sebum production.

FIG. 2 shows the sebum measurement locations. Circle: sebumeter measurement; left square with solid lines: sebutape measurement; right square with broken lines: back-up sebutape measurement.

### DETAILED DESCRIPTION OF THE INVENTION

The specific dosing regimen, optionally together with the specific injection scheme, used in accordance with the present invention allows for a safe and effective treatment with reliable efficacy and predictable results. The treatment safety is improved by reducing toxin spread into nearby muscles, thereby reducing the potential of relevant adverse events in the face, e.g., smile asymmetry, eyebrow and/or eyelid drop. At the same time, due to the relatively high volumes administered, the spread is still sufficient to achieve the desired treatment result.

In addition, the relatively high doses will prolong the duration of effect, thus adding to patient convenience and satisfaction. Also, owing to the specific dosing regimen and injection scheme used herein, involving less injection points and higher volume per injection point, the injection pain is reduced which in turn further improves patient satisfaction. Accordingly, the present invention allows for the efficient and safe treatment of reduction of (enlarged) skin pore size and (excessive) sebum production and leads to a balanced and harmonious appearance of the skin on the upper ⅔ of the face, providing increased patient satisfaction.

The term “skin pore size”, as used herein, refers to the area affected by pores (i.e., the area occupied by the pores within the region of interest expressed, e.g., in mm²) and, as the pores are three-dimensional, to the pore volume (i.e., the overall volume of skin indentations due to the presence of pores in the selected region expressed in, e.g., mm³). Therefore, within the framework of the present invention, it is the area affected by pores and the volume of the pores that get reduced. In contrast, the term “pore count” or “pore number” means the number of individual pores detected in a pre-defined area expressed as a number. The term “skin pore size” in the expression “use of botulinum toxin for the reduction of skin pore size” preferably relates to enlarged skin pore size.

The term “enlarged skin pore size” within the meaning of the present invention may be defined as a skin pore volume that is at least 20%, preferably at least 25%, more preferably at least 30% or at least 35%, and most preferably at least 40% or at least 50% higher than the corresponding skin pore volume in a given region, in particular the cheek and forehead region, measured for a normal (average) subject. For the cheek, assuming that the skin pore volume is 1.25 mm³ per 183 mm² (13.5 mm×13.5 mm) cheek area of a normal subject, an enlarged pore size may be defined as ≥1.50 mm³ per 183 mm² (13.5 mm×13.5 mm) cheek area, preferably ≥1.56 mm³ per 183 mm² cheek area, more preferably ≥1.63 mm³ or ≥1.69 mm³ per 183 mm² cheek area, and most preferably ≥1.75 mm³ or ≥1.88 mm³ per 183 mm² cheek area, as measured for skin pores, i.e. detectable skin pores, having a diameter of between 0.1 mm and 1.0 mm.

For the skin pore measurements, it is preferred to use a camera for image acquisition and corresponding software for analysis of the skin, in particular an Antera 3D imaging system (Miravex Limited, Dublin, Ireland). As used herein, the term “Antera 3D imaging system” is synonymously used with the term “Antera 3D camera” and is intended to refer to a camera and software analysis system of Miravex Ltd. (Dublin, Ireland) for the analysis of the skin. It allows different filters to be applied for defining the cut-off for the detection of a skin feature such as skin pore volume and area. Small filters detect pores that are between 0.1 mm and 0.5 mm in diameter, medium filters detect pores that are between 0.1 mm and 1.0 mm, and large filters detect pores that are between 0.1 mm and 1.5 mm in diameter. Within the present invention, an Antera 3D camera with a medium filter is preferably used for the measurements.

For the forehead, it is assumed herein that the skin pore volume is 1.15 mm³ per 183 mm² (13.5 mm×13.5 mm) for a normal subject. Accordingly, an enlarged pore size (i.e. at least 20%, preferably at least 25%, more preferably at least 30% or at least 35%, and most preferably at least 40% or at least 50% higher) may be defined as ≥1.38 mm³ per 183 mm² (13.5 mm×13.5 mm) forehead area, preferably ≥1.44 mm³ per 183 mm² forehead area, more preferably ≥1.50 mm³ or ≥1.55 mm³ per 183 mm² forehead area, and most preferably ≥1.61 mm³ or ≥1.73 mm³ per 183 mm² forehead area, as measured for skin pores, i.e., detectable skin pores, having a diameter of between 0.1 mm and 1.0 mm.

Alternatively, or in addition to the above definition in terms of the skin pore volume for a given region, the term “enlarged skin pore size” within the meaning of the present invention may, although less preferably, also be defined as an area affected by pores (area occupied by the pores within a given region) that is at least 20%, preferably at least 25%, more preferably at least 30% or at least 35%, and most preferably at least 40% or at least 50% higher than the corresponding area affected by pores in a given region, in particular the cheek and forehead region, measured for a normal (average) subject. For the cheek, assuming that the area affected by pores is 36.6 mm² per 183 mm² (13.5 mm×13.5 mm) cheek area of a normal subject, an enlarged area affected by pores may be defined as ≥43.9 mm² per 183 mm² (13.5 mm×13.5 mm) cheek area, preferably ≥45.8 mm² per 183 mm² cheek area, more preferably ≥47.6 mm² or ≥49.4 mm² per 183 mm² cheek area, and most preferably ≥51.2 mm² or ≥54.9 mm² per 183 mm² cheek area, as measured for skin pores, i.e. detectable skin pores, having a diameter of between 0.1 mm and 1.0 mm.

For the forehead it is assumed herein that the area affected by pores is 33.7 mm² per 183 mm² (13.5 mm×13.5 mm) of the forehead for a normal subject. Accordingly, an enlarged pore size (i.e. at least 20%, preferably at least 25%, more preferably at least 30% or at least 35%, and most preferably at least 40% or at least 50% higher) may also be defined as ≥40.4 mm² per 183 mm² (13.5 mm×13.5 mm) forehead area, preferably ≥42.1 mm² per 183 mm² forehead area, more preferably ≥43.8 mm² or ≥45.5 mm² per 183 mm² forehead area, and most preferably ≥47.2 mm² or ≥50.6 mm² per 183 mm² forehead area, as measured for skin pores, i.e., detectable skin pores, having a diameter of between 0.1 mm and 1.0 mm.

The term “skin pore” or “pore”, as used herein, is intended to refer to depressions of the skin surface that typically have a roundish shape. Skin pores are of relatively large dimensions, being visible to the naked eye. Skin pores within the meaning of the present invention may be defined to have a size ranging from 250 μm to 1000 μm and above, leading to surface areas of 0.05 to 0.8 mm² and above, assuming they have a roundish shape. Macroscopically, they often appear as tiny cuvettes conferring a “golf ball”-like aspect to the skin microrelief. The “skin pore” according to the present invention are not openings or orifices from eccrine sweat glands, which are much smaller in size.

The term “sebum production”, as used herein, means the production of sebum resulting in a certain level (or amount) of sebum on the skin (e.g., expressed in μg/cm²). Preferably, the term “sebum production” used herein refers to “facial sebum production”. The term “sebum”, as used herein, refers to an oily, waxy substance usually comprising triglycerides, wax esters, squalene and other substances that is produced by sebaceous glands.

Sebum supports skin health in several ways, for example it helps to hydrate the skin and protect it from potentially harmful pathogens, such as bacteria and fungi. However, an overproduction or excessive production of sebum can lead to oily skin. People with oily skin may notice that their pores look larger, and their skin appears greasy or shiny. Thus, the term “sebum production”, as used herein within the expression “for the reduction of skin pore size and/or sebum production”, may refer to “excessive sebum production”.

Preferably, the term “excessive sebum production”, as used herein, may be defined as sebum level which is >180 μg/cm² for the forehead, t-zone and scalp, >100 μg/cm² for hair, >150 μg/cm² for the cheek, eyelid and temple, and >130 μg/cm² for the corner of the mouth, as measured at normal room conditions (e.g., 20° C. and 40-60% relative air humidity) by a sebumeter (e.g., Sebumeter® SM 815, Courage+Khazaka). To calculate the absolute amounts of sebum for the full forehead, the value in μg/cm² determined for the forehead may be multiplied by 78 cm², as the typical forehead is 6 cm×13 cm.

It is further noted that the use of botulinum toxin for the reduction of sebum production in accordance with the present invention preferably relates to a purely cosmetic (aesthetic) use, e.g., relates to the treatment of healthy subjects that suffer from increased or excessive sebum production which is undesirable in terms of appearance but is not related with a medical condition or disease. In other words, the use of botulinum toxin for the reduction of sebum production is a cosmetic use for improving the skin quality of (“healthy”) subjects and not the treatment of skin diseases. Specifically, the use of botulinum toxin for the “reduction of sebum production” in accordance with the present invention preferably excludes medical conditions or diseases that involve sebum, in particular acne, sebaceous hyperplasia, steatocystoma (simplex, multiplex), sebaceoma (sebaceous epithelioma, adenoma, carcinoma), and seborrheic dermatitis.

The term “forehead”, as used herein, refers to a facial area that is in its inferior border marked by supraorbital ridge, superior border is the hairline, lateral border is marked by temporal ridge. In addition, the term “cheek”, as used herein, preferably refers to the mid-facial area comprising anterior and medial portion of the cheek (nasolabial and buccal cheek subunit).

The term “subject”, as used herein, is not particularly limited and particularly refers to a human, who is in the need of cosmetic treatment, in particular of the face, more particularly of the upper ⅔ of the face. “Subject” may be interchangeably used with the term “patient” in the context of the present invention. Preferably, the term “subject”, as used herein, relates to a subject having enlarged skin pores size and/or excessive sebum production as defined herein. Particularly preferable, a subject in the context of the present invention is a human having an enlarged pore size corresponding to at least a score of 3 on the Investigator's SASSQ (Scientific Assessment Scale of Skin Quality).

Within the meaning of the present invention, numerical values without decimal places shall be understood to include all numerical values with one or more decimal places that, applying common rules of rounding, give the numerical value without decimal places. For example, the numerical value 3 includes 2.5 or 2.50 (which are rounded up to 3) and 3.4 or 3.49 (which are rounded off to 3), and all numerical values in between. As regards endpoints of ranges, the same applies, i.e., a range of 3 to 5 may mean, e.g., 2.5-3.4 to 4.5-5.4. It is also contemplated that each numerical value or one or both, preferably both, endpoints of ranges disclosed in this application, if they have no decimal places, can be (and replaced by) a numerical value with a decimal place that is zero, e.g., 4 may be 4.0.

The term “botulinum toxin”, as used herein, is not particularly limited and includes botulinum toxin of any serotype (BoNT/A-H), preferably serotypes A and B, either with or without complexing proteins. Hence, within the present invention the term “botulinum toxin” includes a toxin complex comprising the 150 kDa botulinum neurotoxin and complexing proteins (e.g., the 500-900 kDa botulinum toxin complex), or the 150 kDa neurotoxin free of complexing proteins. In the context of the present invention, the botulinum toxin is preferably the 150 kDa neurotoxin free of complexing proteins, particularly of serotype A.

The term “comprising”, like the terms “including” and containing”, and any variations thereof such as “comprises”, “includes” and “contains,”, are intended to refer to a non-exclusive inclusion, such that a process, method, product-by-process, composition or formulation that comprises, includes, or contains an element or list of elements does not include only those elements but can include other elements not expressly listed for such process, method, product-by-process, composition or formulation. In addition, within the framework of the present invention, it is intended that each of the terms “comprise,” “comprising”, “includes”, “including”, “contains”, “containing”, and any variations thereof, can be replaced by the term “consists” or “consisting”, or any variation thereof, which will be understood to refer to an exclusive inclusion of the elements indicated. Furthermore, the terms “a” and “an” and “the” and similar reference used in the context of the present invention are to be construed to cover both the singular and the plural and, thus, may also relate to “at least one” or “more than one”, unless otherwise indicated herein or clearly contradicted by the context.

In a first aspect, the present invention relates to the use of botulinum toxin for the reduction of skin pore size and/or sebum production, wherein the botulinum toxin is administered by intradermal injection into the skin of a subject at a concentration of 11 U/ml to 19 U/ml and at a volume of 0.07 ml to 0.12 ml per injection point.

The botulinum toxin may also be administered at a concentration of 12 U/ml to 19 U/ml, 13 U/ml to 19 U/ml, 14 U/ml to 19 U/ml, 15 U/ml to 19 U/ml, 16 U/ml to 19 U/ml, 17 U/ml to 19 U/ml, 18 U/ml to 19 U/ml, 11 U/ml to 18 U/ml, 12 U/ml to 18 U/ml, 13 U/ml to 18 U/ml, 14 U/ml to 18 U/ml, 15 U/ml to 18 U/ml, 16 U/ml to 18 U/ml, 17 U/ml to 18 U/ml, 11 U/ml to 17 U/ml, 12 U/ml to 17 U/ml, 13 U/ml to 17 U/ml, 14 U/ml to 17 U/ml, 15 U/ml to 17 U/ml, 16 U/ml to 17 U/ml, 11 U/ml to 16 U/ml, 12 U/ml to 16 U/ml, 13 U/ml to 16 U/ml, 14 U/ml to 16 U/ml, 15 U/ml to 16 U/ml, 11 U/ml to 15 U/ml, 12 U/ml to 15 U/ml, 13 U/ml to 15 U/ml, 14 U/ml to 15 U/ml, 11 U/ml to 14 U/ml, 12 U/ml to 14 U/ml, or 13 U/ml to 14 U/ml.

In addition, the botulinum toxin may be administered at a volume of 0.08 ml to 0.12 ml, 0.09 ml to 0.12 ml, 0.10 ml to 0.12 ml, 0.11 ml to 0.12 ml, 0.07 ml to 0.11 ml, 0.08 ml to 0.11 ml, 0.09 ml to 0.11 ml, 0.10 ml to 0.11 ml, 0.07 ml to 0.10 ml, 0.08 ml to 0.10 ml, 0.09 ml to 0.10 ml, 0.07 ml to 0.09 ml, or 0.08 ml to 0.09 ml per injection point.

Furthermore, the botulinum toxin may be administered at a dose of 1.4 U to 1.9 U, 1.5 U to 1.9 U, 1.6 U to 1.9 U, 1.7 U to 1.9 U, 1.8 U to 1.9 U, 1.4 U to 1.8 U, 1.5 U to 1.8 U, 1.6 U to 1.8 U, 1.7 U to 1.8 U, 1.4 U to 1.7 U, or 1.5 U to 1.7 U, 1.5 U to 1.6 U, or 1.6 U to 1.7 U per injection point.

Within the framework of the present invention, it is intended that the concentration ranges of botulinum toxin disclosed herein can be combined with the volumes, the doses, or the volumes and doses of botulinum toxin disclosed herein.

Preferably, the botulinum toxin is administered by intradermal injection at a concentration of 15 U/ml to 19 U/ml and at a volume of 0.075 ml to 0.12 ml per injection point, more preferably at a concentration of 16 U/ml to 19 U/ml and at a volume of 0.075 ml to 0.115 ml per injection point, and most preferably at a concentration of 16 U/ml to 18 U/ml and at a volume of 0.08 ml to 0.115 ml per injection point.

With respect to the injection scheme, the botulinum toxin is generally administered by intradermal injection across the forehead and/or both cheeks of the subject, the forehead comprising the glabellar area. Preferably, the botulinum toxin is administered by intradermal injection into the forehead and both cheeks at a total dose of 30 U to 75 U, preferably 35 U to 65 U, more preferably 40 U to 60 U, and most preferably 45 U to 55 U, the forehead comprising the glabellar area.

The botulinum toxin is administered by intradermal injection into multiple injection points, for example into 12 to 75 injection points, preferably 15 to 60 injection points, more preferably 20 to 50 injection points, and most preferably 25 to 40 injection points, the injection points being distributed across the forehead and both cheeks, the forehead comprising the glabellar area. Particularly preferred within the present invention is the administration of the botulinum toxin by intradermal injection into 7 to 15 injection points, preferably 11 to 13 injection points, across the forehead, the forehead comprising the glabellar area, and into 6 to 12 injection points, preferably 9 injection points, per cheek.

As regards the injection pattern, the injection points are preferably arranged such that the distance between the injection points is 0.9 cm to 1.5 cm, more preferably 1.0 cm to 1.5 cm or 1.1 cm to 1.5 cm, and most preferably 1.2 to 1.4 cm, in the cheek, and 1.7 cm to 2.3 cm, preferably 1.8 cm to 2.2 cm, most preferably 2 cm, in the forehead, the forehead comprising the glabellar area. Within the meaning of the present invention, the term “distance” refers to the distance between any given injection point and another injection point that is closest to said given injection point, unless otherwise stated. It is also contemplated in the present invention that the distance between any two neighbouring injection points does not exceed 1.6 cm to 2.2 cm in the cheek and 2.4 cm to 3.0 cm in the forehead.

Furthermore, according to an especially preferred injection pattern, the botulinum toxin is administered by intradermal injection into 9 injection points per cheek and 7 to 15 injection points, preferably 10 to 12 or 11 to 13 injection points, across the forehead, the forehead comprising the glabellar area, wherein the 9 injection points per cheek are arranged on three horizontal lines and three vertical lines, said lines intersecting each other to define said 9 injection points. In addition, either separately or together with said particularly preferred injection pattern of the cheek, it is particularly preferred that 4 of the injection points of the forehead are located on a first horizontal line and 5 of the injection points of the forehead are located on a second horizontal line that is below the first horizontal line, and optionally wherein 1 injection point is located in the glabellar area.

Particularly preferably, the botulinum toxin is administered by injection into 12 injection points distributed across the forehead, the forehead comprising the glabellar area, wherein 4 of said injection points are located on a first horizontal line and 5 of said injection points are located on a second horizontal line that is below the first horizontal line, and 2 of said injection points are located above the left and right orbital rim (preferably at least 2 cm above the orbital rim) and below said 4 and said 5 injection points, and 1 of said injection points is at the mid-glabellar site.

As used herein, the term “botulinum toxin” is not particularly limited and includes botulinum toxin of any serotype (BoNT/A-H). For example, the botulinum toxin may be of serotype A or B (BONT/A, BONT/B). Preferably, the botulinum toxin is of serotype A, more preferably of serotype A1 (BoNT/A1), and most preferably BoNT/A1 produced by Clostridium botulinum Hall strain. Furthermore, as used herein, the term “botulinum toxin” (“BT”) and the synonymously used term “botulinum neurotoxin” (“BoNT”) are intended to refer to the pure botulinum neurotoxin and/or any complex thereof, i.e., any complex of the pure botulinum neurotoxin and complexing proteins (referred to as the “toxin complex”). Preferably, the botulinum toxin is the pure botulinum neurotoxin of serotype A.

The term “pure botulinum neurotoxin”, as used herein, means the botulinum neurotoxin free of complexing proteins (sometimes also referred to as the “neurotoxic component”), or more precisely, the botulinum neurotoxin without neurotoxin-associated complexing proteins (NAPs). The pure botulinum neurotoxin is the (active) neurotoxic polypeptide that ultimately inhibits acetylcholine release. It is a di-chain protein comprised of a light chain (LC; about 50 kDa) and a heavy chain (HC; about 100 kDa), held together by a disulfide bond. The active neurotoxic polypeptide may therefore also be referred to herein as the “150 kDa neurotoxin”, “Clostridium botulinum neurotoxin (150 kD)” or “neurotoxic component”. Preferably, the botulinum toxin is the (pure) botulinum neurotoxin contained in Xeomin® or is Xeomin®.

The term “toxin complex”, as used herein, refers to a high-molecular complex of the neurotoxic component and a set of complexing proteins (NAPs), including the 900 kDa, 500 kDa, and 300 kDa C. botulinum type A toxin complexes. The complexing proteins are nontoxic nonhaemagglutinin (NTNHA) and, in strains of serotype A-D, different haemagglutinins (HAs). For example, the 900 kDa complex is included in onabotulinumtoxin A (Botox®/Vistabel®, Allergan, Inc., Irvine, CA, USA), and also abobotulinumtoxin A (Dysport®, Azzalure®, Ipsen, Paris, France), Alluzience® (Ipsen/Galderma) and Innotox® (Medytox) contain a toxin complex as active agent. Preferably, the botulinum toxin is the pure botulinum neurotoxin that is contained in Xeomin® or is Xeomin®, or is the toxin complex contained in Botox® or Dysport® or is Botox® or Dysport®.

The botulinum toxin may be a natural neurotoxin obtainable from the bacteria Clostridium botulinum or any other botulinum toxin such as a botulinum toxin obtainable from alternative sources, including recombinant technologies and genetic or chemical modification. Chimeric or genetically modified botulinum toxins, i.e., botulinum toxins containing mutations including substitutions, deletions and insertions, are also encompassed by the terms “botulinum toxin”, “neurotoxic component” and the like. Preferably, the mutation does not compromise any of the biological activities of botulinum toxin. However, it is also envisaged to use mutations to modulate the biological activity of the botulinum toxin. Also included are botulinum toxins containing chemically modified amino acids, for example one or more amino acids which are glycosylated, acetylated or otherwise modified, which may be beneficial to the uptake or stability of the toxin. Particularly preferred is the lipidation of the neurotoxic component.

Within the present invention, the dose is expressed in biological units because the used botulinum toxin may contain, for example, variable percentages of inactive toxin that contribute to the overall protein load without contributing to efficacy. Within the context of the present invention, the biological potency of botulinum toxin is determined using the mouse bioassay (MBA). The MBA determines the mean lethal dose (LD₅₀) of toxin/neurotoxin after intraperitoneal injection in mice, i.e. the dose of toxin/neurotoxin capable of killing 50% of a group of mice. On this basis, 1 unit (U) of toxin/neurotoxin, as used herein, is defined as one mouse LD50 (1.0 LD50=1.0 U). The LD50 mouse bioassay is the gold standard among various biological, chemical or immunological detection methods for botulinum toxin and is known to those skilled in the art (see, e.g., Pearce, L. B.; Borodic, G. E.; First, E. R.; MacCallum, R. D. Measurement of botulinum toxin activity: Evaluation of the lethality assay. Toxicol. Appl. Pharmacol. 1994, 128, 69-77).

Another useful method for determining the biological activity (biological potency) of a botulinum neurotoxin is a cell-based potency assay as it is disclosed, for example, in WO2009/114748, WO 2013/049508 or WO 2014/207109. The activity results obtained with such cell-based assays correspond to the activity values obtained in the mouse i.p. LD50 assay because the values are calibrated using the LD50 reference standard.

Due to differences in the LD50 tests used by manufacturers of commercial botulinum toxin formulations, the unit potencies indicated by the manufacturers for their commercial botulinum toxin formulations is proprietary and cannot easily be compared. Therefore, within the framework of the present invention, the conversion rates provided below are used to establish the comparative potencies of incobotulinumtoxinA (“INCO”; Xeomin®, Bocouture®; botulinum toxin serotype A, free of complexing proteins; Merz Pharmaceuticals GmbH), onabotulinumtoxinA (“ONA”; Botox®, Vistabel®; botulinum toxin complex of serotype A; Allergan Inc.), abobotulinumtoxinA (“ABO”; Dysport®, Azzalure®; botulinum toxin complex of serotype A; Medicis Pharmaceutical Corp., Galderma Lab.), rimabotulinumtoxinB (“RIM”; Myobloc®, NeuroBloc®; botulinum toxin serotype B; Solstice Neurosciences Inc.), and PurTox® (“TBD”; botulinum toxin serotype A; Mentor Worldwide LLC). For use herein, the conversion rate of ONA and INCO is 1:1. The conversion rate of ONA/INCO:ABO is 1:2.5. The conversion rate of ONA/INCO:RIM is 1:50, and the conversion rate of ONA/INCO:TBD is 1:1.5. Furthermore and preferably, within the context of the present invention, 1 U of INCO (Xeomin®) and 1 U of onabotulinumtoxinA (“ONA”; Botox®) shall be deemed to correspond to one mouse LD50 (1.0 LD50), or 1 U, measured as described above.

Generally, the botulinum toxin used within the present invention is in the form of a liquid composition. The liquid composition can be formulated by various techniques dependent on the desired application, as known in the art. It may be provided as a ready-to-use liquid formulation or in the form of a lyophilized powder that is to be reconstituted, typically in physiological saline, prior to use. Preferably, the botulinum toxin used within the present invention is in the form of an aqueous solution, more preferably a saline solution or a physiological saline solution, and most preferably a phosphate buffered physiological saline solution. The aqueous solution may additionally comprise one or more pharmaceutically acceptable substances. Suitable pharmaceutically acceptable substances comprise those well known in the art, see, e.g., Remington's Pharmaceutical Sciences, Mack Publishing Company, Easton, Pennsylvania.

In particular, the aqueous botulinum toxin solution or composition may include other carriers or non-toxic, non-therapeutic, non-immunogenic stabilizers and the like. Thus, the aqueous botulinum toxin composition may contain glycerol, protein stabilizers (HSA) or non-protein stabilizers such as polyvinyl pyrrolidone (PVP), hyaluronic acid or free amino acids, e.g., methionine or histidine. In an aspect, it can be free of amino acids. In an aspect it may be free of stabilizing peptides (e.g., consisting of 5 to 50 amino acids, 10 to 40 amino acids or 15 to 30 amino acids). In an aspect, suitable non-proteinaceous stabilizers are disclosed in WO 2005/007185 or WO 2006/020208. The botulinum toxin composition can also include non-ionic or ionic surfactant, e.g., polysorbate or poloxamer. A suitable formulation for HSA-stabilized formulation comprising a botulinum toxin according to the present invention is for example disclosed in U.S. Pat. No. 8,398,998 B2.

Preferably, the botulinum toxin used within the present invention is in the form of an aqueous solution comprising sodium chloride (NaCl), more preferably in the form of a physiological saline solution (i.e. a solution including sodium chloride in physiological concentration, e.g., about 9 g/l NaCl), wherein the aqueous botulinum toxin solution comprises (i) no other excipient (except NaCl), (ii) human serum albumin (HSA) and a sugar, in particular a monosaccharide or a disaccharide, (iii) human serum albumin (HSA) and lactose, (iv) human serum albumin (HSA) and sucrose, (v) a monosaccharide and/or a disaccharide (e.g. lactose and/or sucrose), (vi) no buffer, (vii) no single amino acids, (viii) no human serum albumin (HSA), sodium chloride and lactose or no HSA, sodium chloride and sucrose, or (ix) no HSA and sodium chloride, or any combination of (i) to (ix).

In a preferred embodiment, the present invention relates to the use of botulinum toxin for the reduction of skin pore size and/or sebum production, wherein the botulinum toxin is administered by intradermal injection into the skin of a subject at a concentration of 14 U/ml to 18 U/ml and at a volume of 0.08 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 14 U/ml to 18 U/ml and at a volume of 0.09 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 14 U/ml to 18 U/ml and at a volume of 0.09 ml to 0.10 ml per injection point.

In a further preferred embodiment, the present invention relates to the use of botulinum toxin for the reduction of skin pore size and/or sebum production, wherein the botulinum toxin is administered by intradermal injection into the skin of a subject at a concentration of 15 U/ml to 19 U/ml and at a volume of 0.08 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 15 U/ml to 19 U/ml and at a volume of 0.09 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 15 U/ml to 19 U/ml and at a volume of 0.09 ml to 0.10 ml per injection point.

In a yet further preferred embodiment, the present invention relates to the use of botulinum toxin for the reduction of skin pore size and/or sebum production, wherein the botulinum toxin is administered by intradermal injection into the skin of a subject at a concentration of 15 U/ml to 18 U/ml and at a volume of 0.08 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 15 U/ml to 18 U/ml and at a volume of 0.09 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 15 U/ml to 18 U/ml and at a volume of 0.09 ml to 0.10 ml per injection point.

In a still further preferred embodiment, the present invention relates to the use of botulinum toxin for the reduction of skin pore size and/or sebum production, wherein the botulinum toxin is administered by intradermal injection into the skin of a subject at a concentration of 16 U/ml to 18 U/ml and at a volume of 0.08 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 16 U/ml to 18 U/ml and at a volume of 0.09 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 16 U/ml to 18 U/ml and at a volume of 0.09 ml to 0.10 ml per injection point.

In a yet still further preferred embodiment, the present invention relates to the use of botulinum toxin for the reduction of skin pore size and/or sebum production, wherein the botulinum toxin is administered by intradermal injection into the skin of a subject at a concentration of 16 U/ml to 17 U/ml and at a volume of 0.08 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 16 U/ml to 17 U/ml and at a volume of 0.09 ml to 0.11 ml per injection point. In another preferred embodiment, the present invention relates to the use of botulinum toxin for the reduction of skin pore size and/or sebum production, wherein the botulinum toxin is administered by intradermal injection into the skin of a subject at a concentration of 16 U/ml to 17 U/ml and at a volume of 0.09 ml to 0.10 ml per injection point.

In a particularly preferred embodiment, the present invention relates to the use of botulinum toxin for the reduction of skin pore size and/or sebum production, wherein the botulinum toxin is administered by intradermal injection into the skin of a subject at a concentration of 14 U/ml to 18 U/ml and at a volume of 0.08 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 14 U/ml to 18 U/ml and at a volume of 0.09 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 14 U/ml to 18 U/ml and at a volume of 0.09 ml to 0.10 ml per injection point and at a dose of 1.4 U to 1.8 U, preferably 1.5 U to 1.8 U, more preferably 1.6 to 1.8 U.

In a further particularly preferred embodiment, the present invention relates to the use of botulinum toxin for the reduction of skin pore size and/or sebum production, wherein the botulinum toxin is administered by intradermal injection into the skin of a subject at a concentration of 15 U/ml to 19 U/ml and at a volume of 0.08 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 15 U/ml to 19 U/ml and at a volume of 0.09 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 15 U/ml to 19 U/ml and at a volume of 0.09 ml to 0.10 ml per injection point and at a dose of 1.4 U to 1.9 U, preferably 1.5 U to 1.9 U, more preferably 1.5 to 1.8 U.

In a yet further particularly preferred embodiment, the present invention relates to the use of botulinum toxin for the reduction of skin pore size and/or sebum production, wherein the botulinum toxin is administered by intradermal injection into the skin of a subject at a concentration of 15 U/ml to 18 U/ml and at a volume of 0.08 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 15 U/ml to 18 U/ml and at a volume of 0.09 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 15 U/ml to 18 U/ml and at a volume of 0.09 ml to 0.10 ml per injection point and at a dose of 1.4 U to 1.8 U, preferably 1.5 U to 1.8 U, more preferably 1.6 to 1.8 U.

In a still further particularly preferred embodiment, the present invention relates to the use of botulinum toxin for the reduction of skin pore size and/or sebum production, wherein the botulinum toxin is administered by intradermal injection into the skin of a subject at a concentration of 16 U/ml to 18 U/ml and at a volume of 0.08 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 16 U/ml to 18 U/ml and at a volume of 0.09 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 16 U/ml to 18 U/ml and at a volume of 0.09 ml to 0.10 ml per injection point and at a dose of 1.4 U to 1.8 U, preferably 1.5 U to 1.8 U, more preferably 1.6 to 1.8 U.

In a yet still further particularly preferred embodiment, the present invention relates to the use of botulinum toxin for the reduction of skin pore size and/or sebum production, wherein the botulinum toxin is administered by intradermal injection into the skin of a subject at a concentration of 16 U/ml to 17 U/ml and at a volume of 0.08 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 16 U/ml to 17 U/ml and at a volume of 0.09 ml to 0.11 ml per injection point and at a dose of 1.4 U to 1.9 U, preferably 1.5 U to 1.9 U, more preferably 1.5 to 1.8 U. In another particularly preferred embodiment, the present invention relates to the use of botulinum toxin for the reduction of skin pore size and/or sebum production, wherein the botulinum toxin is administered by intradermal injection into the skin of a subject at a concentration of 16 U/ml to 17 U/ml and at a volume of 0.09 ml to 0.10 ml per injection point and at a dose of 1.4 U to 1.7 U, preferably 1.5 U to 1.7 U, more preferably 1.6 to 1.7 U.

In a yet another particularly preferred embodiment, the present invention relates to the use of botulinum toxin for the reduction of skin pore size and/or sebum production, wherein the botulinum toxin is administered by intradermal injection into the skin of a subject at a concentration of 16.67 U/ml and at a volume of 0.10 ml per injection point and at a dose of 1.67 U.

Moreover, it is contemplated herein, in particular with regard to the preferred and particularly preferred embodiments described in the preceding eleven paragraphs, that the botulinum toxin is preferably administered by intradermal injection across the forehead and/or both cheeks, preferably across both cheeks and the forehead, of the subject, the forehead comprising the glabellar area. Furthermore, if the botulinum toxin is administered by intradermal injection across (into) both cheeks and forehead (the forehead comprising the glabellar area), the total dose of botulinum toxin administered is preferably 30 U to 75 U or 35 U to 65 U, more preferably 40 U to 60 U, and most preferably 45 U to 55 U.

In a second aspect, the present invention relates to a method for the reduction of skin pores and/or sebum production, the method comprising administering botulinum toxin by intradermal injection into the skin of a subject at a concentration of 11 U/ml to 19 U/ml and at a volume of 0.07 ml to 0.12 ml per injection point.

This method of treatment may be used for purely aesthetic purposes, i.e., for improving certain skin quality attributes to improve the overall visual appearance of the skin.

The method according to the second aspect of the present invention is closely related to the use according to the first aspect of the present invention. Thus, all definitions, explanations, advantages etc. given herein with respect to the use according to the first aspect equally apply to the method according to the second aspect.

The method according to the second aspect of the present invention is closely related to the use according to the first aspect of the present invention. Thus, all definitions, explanations and advantages given herein with respect to the use according to the first aspect equally apply to the method according to the second aspect.

### EXAMPLES

### Example 1

### Dosing Regimen and Injection Scheme According to the Present Invention

A particularly preferred treatment regimen (i.e., dosing regimen and injection scheme) for the reduction of enlarged skin pore size and/or excessive sebum production involves the reconstitution of a 100 U vial (e.g., of NT 201 (Xeomin®)) with 6 ml saline (0.9% w/v NaCl solution), resulting in the concentration of 16.67 U/ml. Alternatively, a 50 U vial can be reconstituted with 3 ml saline.

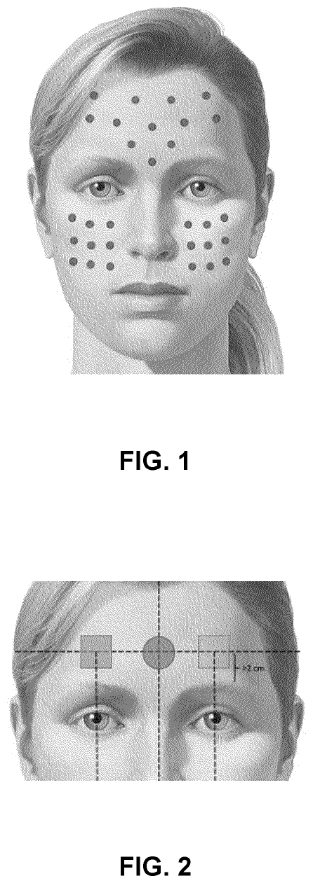

The reconstituted botulinum toxin formulation of 16.67 U/ml is then intradermally injected in 30 injection points distributed across the forehead and both cheeks of a subject as follows: 12 injection points on the forehead (each 100 μl/1.67 U) and 9 injection points per cheek (18 in total; each 100 μl/1.67 U). The injection pattern is shown in FIG. 1.

As can be seen from the figure, the distance between the injection points is about 2 cm on the forehead and about 1 to 1.5 cm in the cheek area (depending on the facial characteristics, surface area and size of patient's head). Any suitable injection device, such as a 0.5-1.0 ml syringe equipped with a 30 G to 33 G needle (e.g., a 32 G or 33 G needle), may be used to administer the botulinum toxin solution by intradermal injection.

The described combination of reconstitution volume, injection dose/volume and injection scheme results in an effective treatment of enlarged pores and excessive sebum production in the upper ⅔ of the face, enhancing patient satisfaction without increasing side effects.

### Example 2

### Study to Investigate the Efficacy and Safety of Botulinum Toxin in the Treatment of Enlarged Pores and Excessive Sebum Production

A typical study to investigate the efficacy and safety of botulinum toxin in the treatment of enlarged pores and excessive sebum production in the face is described in the following. A total of, e.g., 30 subjects (male or female) is included in a randomized, double-blind, placebo-controlled study. The main inclusion criteria are enlarged pore size (e.g., at least large score of, e.g., ≥3, on Investigator's SASSQ (Scientific Assessment Scale of Skin Quality) and oily skin on the forehead (e.g., >180 μg/cm², as measured by sebumetry), aged, e.g., 18-50 or 21-39 years. Among others, subjects with botulinum toxin treatments and other facial cosmetic treatments (e.g., laser or ultrasound treatment) within the last 12 months are not included in the study.

The total number of 30 subjects are randomized to two different treatment groups with a randomization of, e.g., 2:1 (botulinum toxin:placebo group). All subjects are injected intradermally with a saline solution (0.9% sodium chloride) of botulinum toxin (e.g., NT 201, incobotulinumtoxinA) or placebo in the upper face (forehead and cheek area) using, e.g., the dosing regimen and injection scheme described in Example 1. The placebo is a placebo solution (0.9% sodium chloride) only containing the excipients of the used saline solution of botulinum toxin (i.e., sucrose and human serum albumin in case of NT 201).

All randomized subjects are then followed for a period of, e.g., 90 days (injection session at day 1). Efficacy variables are determined at baseline (day 1) and regular follow-ups at, e.g., 1 week, 4 weeks, 8 weeks and 12 weeks to assess the reduction in pore size (e.g., in the present study pore volume) and sebum production. In addition, safety is assessed using safety parameters including Adverse Events (AEs), including AEs of Special Interest (AESIs) and serious AEs (SAEs). Specific efficacy variables suitable for assessing the efficacy of botulinum toxin for reducing pore size and sebum production are explained below.

Pore volume. The pore volume is the overall volume of skin indentations due to the presence of pores in the selected region expressed in mm³. In this study, the absolute change in pore volume from baseline to a certain point in time, e.g., week 4 (primary outcome variable) is measured in the left and right cheek areas and the forehead using a camera for image acquisition and corresponding software for analysis of the skin. In particular, an Antera3D (Miravex Limited, Dublin, Ireland) camera and corresponding skin analysis software can be used for this purpose (see, e.g., Messaraa et al., Antera 3D capabilities for pore measurements, Skin Res. Technol. 2018; 24 (4):606-613). Triplicate measurements are performed on the cheek and forehead area by placing the camera directly onto the skin without excessive pressure using a medium filter. On the cheek the central area of 900 mm² (30×30 mm²) is analyzed. On the forehead, full field is analyzed, i.e., an area of 3136 mm² (56×56 mm²). In addition to pore volume, the following pore parameters may be assessed and analyzed: pore count (the number of individual pores detected in a pre-defined area expressed as a number) and the area affected by pores (area occupied by the pores within the region of interest expressed in mm²).

A subject with normal skin has a pore volume of about 1.25 mm³, (and pore count of about 54) measured for a central cheek area of 13.5 mm×13.5 mm (183 mm²) using Antera 3D with medium filter. Assuming that the pore volume of a subject with enlarged pores in the same area (183 mm²) exceeds the value of 1.25 mm³ by at least 20%, enlarged pore size (pore volume) can be defined as, e.g., ≥1.50 mm³ per 183 mm² cheek area. Taking into account that the cheek assessment area as defined above covers 900 mm², the baseline pore volume of a subject with enlarged pores in the cheek area as measured by Antera3D using medium filter is estimated to be about 7.4 mm³.

Sebum production. The lipid layer on the surface of the skin consists of epidermal lipids produced by the sebaceous glands. The amount of sebum on the surface of the skin is an important factor in assessing the condition of the skin and provides information about the number and activity of the sebaceous glands in the area examined. In this study, the time course of sebum level [μg/cm²] in the forehead and changes from baseline are measured by sebumetry using a sebumeter (e.g., (Sebumeter® SM 815, Courage+Khazaka). To this end, the sebum is collected from the skin surface on the midline forehead (see FIG. 2) with a semi-transparent plastic strip in a measuring area of, e.g., 64 mm² and the value is expressed in μg/cm². Midline forehead is defined as approximately 2 cm superior to the subject's glabella. Excessive sebum production on the forehead (“oily skin”) is defined as a value of >180 μg/cm².

Sebum composition. The changes in sebum composition in the forehead is measured using Mass Spectroscopy as known in the art. The following lipid classes are analyzed: triglycerides, squalene, wax esters, free fatty acids, and cholesterol. For sebum composition analysis, sebum samples are collected from the skin surface with a sebum absorbent tape (SebuFix, Courage+Khazaka) on the left or right side of the forehead defined as mid forehead above the midpupillary line. If any technical error occurs during application of the tape to the forehead or transfer to the container rendering it invalid, an additional tape may be collected on the opposite side of the mid forehead above the midpupillary line (see FIG. 2 for sebum measurement locations). Sebutapes are applied on the forehead for 10 minutes and collect sebum from an area of about 4 cm². Sebutapes are transferred to a sterile container, optionally stored at least at −20° C. (optimally at −80° C.), and then subjected to lipidomic analyses using Mass Spectroscopy.

Skin oiliness. In addition, skin oiliness is determined using the SOS (Skin Oiliness Scale). SOS is a validated, six-point scale that correlates with the Sebumeter measurements (Baumann et al., A Validated Questionnaire for Quantifying Skin Oiliness, J. Cosmet. Dermatol. Sci. Appl., 2014, 4, 78-84). The SOS is assessed by a questionnaire to accurately determine skin oiliness and can be used in screening and recruiting patients for the study. The change of SOS over time in the present study is to be assessed by the subjects.

Subject's and Investigator's SASSQ. The SASSQ (Scientific Assessment Scale of Skin Quality) is a visual, standardized, scientific assessment tool for measuring skin quality of human skin. In this study, the time course of SASSQ for pore size, wrinkles, skin surface roughness, erythema and blemishes is assessed by the subjects and investigators and changes from baseline are determined. The five image grades of the scales show the evident severity of aging processes of these changes. The response options of the scale are 0 for none, 1 for mild, 2 for moderate, 3 for severe, and 4 for very severe changes. The SASSQ divides the face in 11 face-clusters (forehead, glabella, right eye, left eye, left cheek, nose, right cheek, perioral area, left jawline area, chin area, right jawline area). The face-clusters used in this study may be, e.g., forehead, glabella, left cheek, right cheek.

Investigator's and subject's GAIS. GAIS (global aesthetic improvement scale) is a balanced 7-point Likert scale (range −3 to +3) commonly used in aesthetic medicine to rate global aesthetic improvement in appearance after treatment. A global assessment of aesthetic improvement in appearance of pores (size and volume) and sebum production compared to the status prior to the injection as documented in baseline photographs are individually assessed by the investigator and the subjects using the GAIS starting from, e.g., week 1.

FACE-Q. The FACE-Q© is a patient-reported outcome (PRO) measure that can be used to measure outcomes of aesthetic facial procedures and products from the patient's perspective. Each FACE-Q© scale is composed of a series of items (or questions) that evaluate a concept of interest. Patient responses to items are on a four-point scale and are asked to answer with their face/facial appearance in mind. Answers for a scale are summed and converted to a score from 0 to 100. For most FACE-QC scales, a higher score indicates a better outcome. The following FACE-Q questionnaires may be used in this study: FACE-Q Satisfaction with skin (appearance of facial skin), FACE-Q Satisfaction with facial appearance (appearance of the entire face), FACE-Q Aging appraisal (how a respondent feels about the age his/her face looks), FACE-Q Age appraisal visual analogue scale (rating of perceived age in comparison to chronological age), FACE-Q Appearance-related psychosocial distress (appearance-related distress), and FACE-Q Early life impact of treatment (how a respondent feels after an aesthetic treatment).

The primary outcome variable is the change in pore size, more specifically pore volume, from baseline. Another key outcome variable is the reduction in sebum production over baseline. The statistical analysis of the data demonstrates a significant reduction of pore volume and sebum production.

1-15. (canceled)

16. A method for reduction of skin pore size and/or sebum production, the method comprising administering botulinum toxin by intradermal injection into skin of a subject at a concentration of 11 U/ml to 19 U/ml and at a volume of 0.07 ml to 0.12 ml per injection point.

17. The method of claim 16, wherein the botulinum toxin is administered at a concentration of 13 U/ml to 19 U/ml.

18. The method of claim 16, wherein the botulinum toxin is administered at a concentration of 16 U/ml to 18 U/ml.

19. The method of claim 16, wherein the botulinum toxin is administered at a volume of 0.08 ml to 0.12 ml per injection point.

20. The method of claim 16, wherein the botulinum toxin is administered at a volume of 0.10 ml to 0.11 ml per injection point.

21. The method of claim 16, wherein the botulinum toxin is administered at a dose of 1.4 U to 1.9 U per injection point.

22. The method of claim 16, wherein the botulinum toxin is administered at a dose of 1.6 U to 1.8 U per injection point.

23. The method of claim 16, wherein the botulinum toxin is administered by intradermal injection at a concentration of 15 U/ml to 19 U/ml and at a volume of 0.075 ml to 0.12 ml per injection point.

24. The method of claim 16, wherein the botulinum toxin is administered by intradermal injection across the forehead and/or both cheeks of the subject, the forehead comprising the glabellar area.

25. The method of claim 16, wherein the botulinum toxin is administered by intradermal injection into the forehead and both cheeks at a total dose of 30 U to 75 U.

26. The method of claim 16, wherein the botulinum toxin is administered by intradermal injection into the forehead and both cheeks at a total dose of 45 U to 55 U.

27. The method of claim 16, wherein the botulinum toxin is administered by intradermal injection into 12 to 75 injection points, the injection points being distributed across the forehead and both cheeks, the forehead comprising the glabellar area.

28. The method of claim 16, wherein the botulinum toxin is administered by intradermal injection into 25 to 40 injection points, the injection points being distributed across the forehead and both cheeks, the forehead comprising the glabellar area.

29. The method of claim 16, wherein the botulinum toxin is administered by intradermal injection into 7 to 15 injection points across the forehead, the forehead comprising the glabellar area, and into 6 to 12 injection points per cheek.

30. The method of claim 16, wherein the botulinum toxin is administered by intradermal injection into 11 to 13 injection points across the forehead, the forehead comprising the glabellar area, and into 9 injection points per cheek.

31. The method of claim 16, wherein the botulinum toxin is administered by intradermal injection into 9 injection points per cheek and 7 to 15 injection points across the forehead, the forehead comprising the glabellar area, and wherein the 9 injection points per cheek are arranged on three horizontal lines and three vertical lines, said lines intersecting each other to define said 9 injection points.

32. The method of claim 31, wherein 4 of the injection points of the forehead are located on a first horizontal line and 5 of the injection points of the forehead are located on a second horizontal line that is below the first horizontal line.

33. The method of claim 32, wherein the botulinum toxin is administered by injection into 12 injection points distributed across the forehead, the forehead comprising the glabellar area, wherein 4 of said injection points are located on a first horizontal line and 5 of said injection points are located on a second horizontal line that is below the first horizontal line, and 2 of said injection points are located above the left and right orbital rim and below said 4 and said 5 injection points, and 1 of said injection points is at the mid-glabellar site.

34. The method of claim 27, wherein the shortest distance between the injection points is 0.9 cm to 1.5 cm in the cheek and 1.7 cm to 2.3 cm in the forehead, the forehead comprising the glabellar area.

35. The method of claim 31, wherein the botulinum toxin is a toxin complex of the 150 kDa botulinum neurotoxin and complexing proteins or is in the form of the 150 kDa neurotoxin free of complexing proteins and/or wherein the botulinum toxin is of serotype A or B.
